(19) United States
(12) Patent Application Publication
(10) Pub. No.: US 2003/0143311 A1
(43) Pub. Date: Jul. 31, 2003

(54) Recovery drink formula and method

(76) Inventor: William Gillota, Tequesta, FL (US)

Correspondence Address: REED SMITH CROSBY HEAFEY LLP, 1901 AVENUE OF THE STARS, SUITE 700, LOS ANGELES, CA, 90067, US

(21) Appl. No.: 10/278,678

(22) Filed: Oct. 23, 2002

Related U.S. Application Data
(60) Provisional application No. 60/339,407, filed on Oct. 26, 2001.

(51) Int. Cl. A23L 2/00; A61K 31/70; A61K 31/525; A61K 31/714

(52) U.S. Cl. 426/590; 424/439; 514/23; 514/52; 514/251

(57) ABSTRACT

The present invention is a recovery drink formula and method which safely provides individuals with enhanced, faster, and more complete athletic recovery. The present invention is made from natural ingredients, mostly vitamins and minerals. The present invention may be made in the form of a powder, which is later added to water, or can be made directly as a liquid drink. The drink provides nutrients to ensure recovery following strenuous physical activity.

The present application is based on and claims priority from U.S. Application No. 60/339,407, filed Oct. 26, 2001, which is specifically incorporated herein by reference.

### BACKGROUND OF THE INVENTION

1. Area of the Art

The present invention is a nutritional drink formula and method. More particularly, the present invention is a sports drink designed to improve personal athletic performance. This unique recovery drink is specifically designed and engineered to improve athletic performance during physical activity, to minimize the debilitating effects of dehydration and to maximize post-performance recovery.

2. Description of the Prior Art

In recent times, more and more people are partaking in athletic activities of all types and at all levels. This trend is true of all facets of the population including children, adults, blue collar workers, professionals, men, women, and so on.

Given that the United States is one of the hardest working countries in the world, stamina required for athletic activity can sometimes wane. And while many attempt selective diets, vitamins, and stimulants (such as coffee or tea), to increase their athletic prowess, there is no one remedy to assure quick and full recovery after athletic activity. This is particularly true given today's focus on natural, holistic products.

A number of inventions exist which attempt to address the needs of those who perform athletic activity, but none as does the present invention. For example, the reference to Langer et al., U.S. Pat. No. 4,737,367, discloses an improved, tasty fruit-flavored beverage which contains all, or substantially all, of the U.S. Recommended Daily Allowance of vitamins. This drink is a combination of orange, pineapple, apple, grape, pear, apricot, peach, nectarine, and banana puree designed to replenish lost vitamins; however, this reference does not provide energy or stamina for athletic activity, nor direct recovery benefits for post-exercise activities.

The reference to Schechter, U.S. Pat. No. 6,071,547, discloses a dry mix formulation for a nutritional drink that contains, among other ingredients, an optional vitamin-mineral mixture. Once again, while this invention is designed to replenish lost nutrients, it does not provide increased stamina or enhanced athletic recovery rates.

The reference to Fratzer, U.S. Pat. No. 4,874,603 discloses the use of Vitamin E for normalization of blood coagulation. While this reference provides medical benefits to patients having blood coagulation problems, it is not designed to increase stamina and recovery associated with athletic activity.

Similarly, the reference to Baumann, U.S. Pat. No. 6,103,755, utilizes vitamins in a foodstuff to ameliorate cardiovascular diseases and cancer. Once again, while its invention utilizes vitamins to address health conditions, it is not intended for enhanced athletic recovery.

Finally, the reference to Atkinson, et al. U.S. Pat. No. 6,207,203, discloses a fortified coffee drink providing protein, vitamins and minerals, in addition to caffeine. While this invention is alleged to increase user awareness, athletic recovery is not contemplated. Indeed, the use of caffeine during or after athletic activity may inhibit, rather than enhance, recovery and quite possibly could compromise health.

The prior art is attempting to solve the problem of beverages designed for physical activity with a “one size fits all” approach. The present inventor has determined that there are really two aspects to the problem. The athlete requires a beverage to replenish water, salts and energy during physical activity to sustain energy and help prevent soreness and other training injuries. Such an “energy drink” should also contain vitamins and other biological factors to aid in absorption, etc. However, such a beverage cannot have either the nutrient or biological factor levels necessary to ensure optimal recovery and stamina for later exercise because the required levels would impede absorption during exercise and would negatively affect the blood sugar. Therefore, it is necessary to provide one beverage for use during exercise and a second “recovery” beverage for use between exercise episodes. The instant invention addresses such a recovery beverage.

### SUMMARY OF THE INVENTION

The present invention provides a unique formula for replenishing energy stores and for overall recovery following strenuous activity. A 32-ounce serving provides energy, hydration, minerals and nutrients in levels appropriate for recovery and replenishing glycogen stores. The formula provides carbohydrate energy from glucose polymers (maltodextrins), glucose (dextrose) and sucrose (approximately 4-12 calories per ounce). The glucose polymers help ensure sustained blood sugar levels, whereas similarly high levels of glucose alone would result in more rapid peaking of blood sugar followed by a fall in blood sugar. The maltodextrins represent at least about one half or more of the carbohydrates while glucose represents the majority of the carbohydrate not provided by maltodextrin. A combination of straight chain and branched chain (with leucine dominant) amino acids supplement the carbohydrates for energy production in muscle tissue during exercise and for tissue repair following exercise. Straight-chain amino acids (glutamine and glycine) and essential branched-chain amino acids are provided. The molar ratio of glycine to glutamine to valine to isoleucine to leucine of approximately 2:5:1:1:3 (molar ratio of about 1.5:1 straight chain to branched chain amino acids), are provided to supplement glucose for energy production in muscle tissue. The weight ratio of carbohydrates to total amino acids is between about 30:1 and 60:1. Between 100 mg and 300 mg of calcium (from calcium carbonate) is provided per serving. Between about 60 mg and 180 mg of magnesium is also provided with the weight ratio of calcium to magnesium is between about 3:1 and 1:1. The weight ratio of calcium to zinc is between about 30:1 and 10:1. It is believed that the hypotonic combination of salts (calcium, magnesium, zinc, sodium and potassium at an approximate weight ration of 20:12:1:20:30) and glucose provides for rapid and painless absorption of water, salts and carbohydrate. In addition chromium is present at about between 50% and 150% of the recommended daily value. Vitamins, ensure maximum metabolic activity for energy production and tissue growth. Approximately 10%-300% percent of the recommended daily requirement of Vitamins B₁₂ and folate are provided per serving along with between about 50% and 1,000% of the recommended daily value for vitamins B₁ (thiamine) and B₆. In addition, between 50% and 600% of the recommended daily values of vitamin C and vitamin E are provided. Between 10% and 300% of vitamin A (as retinyl acetate) is also provided. Vitamins also facilitate metabolism and provide significant antioxidants (both water and lipid soluble). The antioxidant effect is greatly increased Green Tea Extract (between about 5 and about 500 mg), Grape Seed Extract (between about 5 and about 500 mg) and Ferulic Acid (between about 5 and about 500 mg). Both Korean Ginseng (about 25 to about 200 mg) and Siberian Ginseng (about 25 to about 200 mg) are supplied to enhance energy levels. Ornithine α-ketoglutarate (between about 100 and about 1,000 mg) is included to stimulate recovery and tissue building. Glucosamine is provided for the long-term condition of connective tissue and joint lubricity. Glucosamine is present at a molar ratio to free glucose of between about 20:1 and about 120:1.

### DESCRIPTION OF THE FIGURES

FIG. 1 is a table listing the ingredients in an embodiment of the inventive composition. Serving size is 32 ounces and daily value (DV) is based on a 2000-calorie diet.

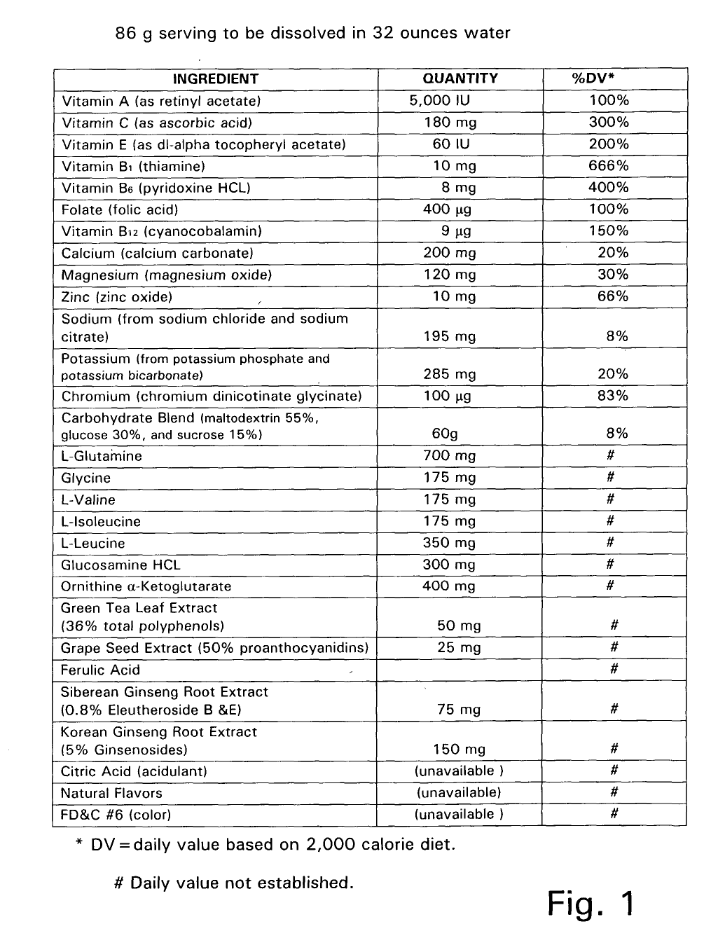

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide a beverage optimized to ensure recovery from strenuous exercise so that subsequent athletic performances will be enhanced rather than impaired.

Embodiments consistent with the present invention address the need for an efficient recovery formula and method that provides the consuming public with a technique to assist and maximize post-performance recovery. While the prior art attempts to address this need, only the present invention is able to accomplish this goal. The present invention, described herein, may be implemented over a variety of platforms. Nevertheless, for the purposes of setting forth the preferred embodiment, the present invention is described with regard to a drink formula.

Product Description

The present invention is a unique recovery drink specifically designed and engineered to improve post-athletic performance recovery. The present invention provides a nutritionally balanced and supplemented recovery drink for the training room and athletic field that will greatly improve personal performance. The product provides adequate calories to avoid the weight loss that often follows strenuous exercise. The product also provides amino acids, vitamins and other biological factors so that optimal recovery and repair as well as new muscle growth occur.

Glossary

These terms have the following meanings herein:

Acidulant—An acid compound (Citric Acid) used to modify the flavor and or aid in the preservation of a food product by lowering the pH.

Aerobic—With oxygen.

Aerobic activity—A low-intensity, high endurance activity that requires oxygen for endurance.

Amino Acid—The linking together molecules of protein (linked though peptide bonds) that forms the building blocks for the cells of the body.

Anabolic—The biochemical process in which different molecule combine to form larger, more complex molecules.

Anabolism—The biochemical process in which different molecule combine to form larger, more complex molecules.

Anaerobic—Without oxygen.

Anaerobic activity—A high intensity, low endurance activity that requires bursts of energy for power and speed.

Anion—A negatively charged ion (e.g., chloride).

Anticatabolic—Describing a substance that prevents the catabolic process (the break down of complex molecules) from occurring.

Antioxidant—A nutrient that has been found to seek out and neutralize free radicals in the body, generally prevent inappropriate oxidation of cellular components and thereby help the body to recover more quickly from the damage caused by free radicals and oxidative stress.

BCAA—Branched chain amino acids L-Isoleucine, L-Leucine, and L-Valine. These make up about 35 percent of the total amino acids in muscle tissue.

Bioavailability—The ability of an ingested nutrient to cross from the digestive tract into the bloodstream and then from the bloodstream into the cells in which it will be utilized.

Bioflavonoids—A group of naturally occurring antioxidant plant compounds that help maintain and strengthen the artery walls of the circulatory system.

Carotenoids—A class of chemical that displays Vitamin A activity. They are made by plants and are converted to Vitamin A within the body.

Catabolism—The biochemical process in which complex molecules are broken down for energy production, recycling of their components, or excretion.

Cation—A positively charged ion.

Coenzyme—An enzyme cofactor or substance needed for an enzyme to perform its function.

Cofactor—A substance that must be present for an enzyme to be able to perform a specific function.

Collagen—A structural protein that is the chief component of connective tissue.

Complex carbohydrates—A polysaccharide (carbohydrate that has 3 or more sugar molecules bound together) such as starch, dextrin, cellulose, and glycogen (which are all glucose polymers).

Cortisol—A hormone secreted by the adrenal glands that stimulates the breakdown of complex molecules for energy.

DNA—Deoxyribonucleic acid. The substance in the cell's nucleus that contains the cell's genetic blueprint and determines the sequence of all the proteins made by the cell.

Epithelial cells—The type of cells that compose such structures as the linings of the lungs and digestive system.

Ergogenic—A catchall term that describes anything that can be used to enhance athletic performance. Ergogenic aids can be dietary or non dietary and include dietary supplements, special training techniques, and mental strategies.

Free radical—One of the highly reactive molecules that are known to injure cell membranes and other cellular components, cause damage to DNA and contribute to the aging process and a number of other degenerative diseases and illnesses. Free radicals are unintended byproducts of normal chemical reactions in the body that involve oxygen.

GABA—Gamma-aminobutyric acid an amino acid and neurotransmitter that is calming to the brain. This calming effect can be beneficial to athletes who require concentration or steadiness. It can also assist athletes who are effected by stress.

GH—Growth hormone.

Hemoglobin—The red protein that carries oxygen in red blood cells.

Krebs Cycle—The metabolic process by which energy is released from glucose, fatty acid or protein molecules and used to regenerate adenosine triphosphate (ATP) molecules (also known as the tricarboxylic acid cycle).

Lactic acid—A toxic byproduct of anaerobic glucose metabolism.

Neurotransmitter—A chemical substance that aids in the transmission of nerve impulses.

Nitrosamines—Organic compounds present in various foods and found to produce cancer in laboratory animals.

Osmotically balanced—A combination of ions that enhances the body's cellular ability to efficiently and effectively absorb the ingredients and nutrients.

Oxidation—A chemical reaction in which an atom or molecule loses electrons.

pH—Potential of hydrogen. The negative logarithm of the concentration of hydrogen ions in a solution—numbers below 7 indicate an acid condition.

Phenols—Often toxic organic chemicals detoxified in the liver and bound in the form of bile salts.

Phenylalanine—Is an essential amino acid and is the precursor of several important metabolites such as the skin pigment melanin.

Phospholipids—A macronutrient. Phospholipids are the second major class of lipids. They are a major structural part of every living cell where they make up the cell membranes.

Potentiator—A substance that helps another substance perform its function.

Prostanoid—A derivative of the hormone prostaglandin, which is important in metabolism and in the reproductive process, and blood-platelet aggregation.

Reversed engineered—Starting with desired performance criteria, then selecting ingredients and quantities that create the desired results/effects based upon scientific research.

RNA—Ribonucleic acid. The substance that carries the coded genetic information from the DNA, in the cell nucleus, to the ribosome's, where the instructions are translated into the form of protein molecules.

Synthesize—To form or make up.

Tyrosine—An amino acid. The precursor of nonrepinephrine and dopamine, regulates appetite, and aids in melanin production.

Uptake—Absorption, as in absorption by the cells and into the cells.

The present invention is a unique recovery drink specifically designed and engineered to improve athletic performance by providing nutrients to help recovery and rebuilding between bouts of physical activity. The present invention provides a nutritionally balanced and supplemented recovery drink for the training room and athletic field that can significantly improve personal performance. The present invention is designed to assist the body in achieving optimum performance levels by ensuring optimum recovery between episodes of exercise. For overall support of athletic training it is believed that intake of adequate water, calories, amino acids, minerals and other nutrients are necessary. Supplement drinks are beneficial because a typical diet may not provide all of the required factors. However, a single supplement drink is not the overall answer because a drink providing sufficient nutrients and calories for overall training recovery is not compatible with the need for energy and hydration during strenuous exercise. Rather, the inventor has divided the overall requirements into an energy drink for use during physical activity and a recovery drink for use between exercise bouts. The present invention concerns the second of these two complementary beverages.

The present invention sustains physical exercise and contains special ingredients to provide, directly or indirectly, benefits that include:

Promoting the development of lean, strong efficient muscle mass.

Increasing total physical strength.

Extending physical endurance.

Improving flexibility.

Enhancing agility.

Increasing speed.

Improving circulation.

Increasing anaerobic capacity and endurance.

Raising individual performance levels and results.

Physical Properties

The present invention is a non-effervescent powder that is mixed with water. This powder is a coated granular mix. When the powder is mixed with water, it becomes a hypotonic solution. The powder is mixed with 32 ounces of water for individual dosage/serving. The formula can also be premixed with water and sold as a liquid. The resulting mixture dissolves in tap water without clumping, floating or settling, and it must dissolve and remain in a clear transparent solution. Because of the high level of vitamins and other nutrients optimal solubility is best achieved by mixing a single serving of the powder mix (86 g) with about five ounces of hot tap water (120-150° F.). After initial vigorous mixing to ensure hydration of the components, 27 ounces of cold or iced water can then be added to bring the mixture to its final dilution.

The present invention does not have or leave residue on the top, sides or bottom of the mixing or delivery vessel. In the preferred embodiment, the present invention is lightly orange flavored, not too sweet, slightly saline.

Ingredients

The present invention is a glucose based complex carbohydrate formula. Carbohydrates are important source because they most effectively refill the glycogen stores in the muscles and liver. In addition, they elevate the blood sugar to a level sufficient for long sessions of intensive training. If the athlete's energy stores become drastically depleted or they experience lactic-acid build up, they will suffer temporary muscle fatigue.

The present invention contains the following ingredients in balanced and meaningful quantities.

Vitamins A, C, E and B₁, B₆, B₁₂ and Folate

Vitamin A, a fat-soluble vitamin is essential for maintenance of vision and proper cellular growth and development. It is needed for the proper maintenance and integrity of the immune system; for the formation and maintenance of healthy skin, hair, and mucous membranes; as well as for bone growth and tooth development.

Vitamin C is primarily a water-soluble antioxidant that cannot be synthesized by the human body. It is involved in the formation and maintenance of collagen, a protein that is an important component of skin, ligaments, and bones. It helps heal wounds, may help fight infections, and promotes healthy capillaries, gums and teeth.

Vitamin E, another fat-soluble vitamin, assists in red blood cell formation, acts as an antioxidant, and aids in the regulation of prostanoid synthesis. Prostanoids are compounds that are important in the reproductive process, and blood-platelet aggregation (clustering). Of interest to athletes, Vitamin E has also been shown to lower the blood-lactate level, decrease the formation of certain waste products during exercise, reduce oxidative cellular damage, maintain muscle tissue, and possibly help testosterone production.

Vitamin B1 (thiamine) is converted by the body into coenzymes that are essential for carbohydrate metabolism and proper nerve functioning. Since athletes need to rapidly metabolize more carbohydrates than non-athletes do, their need for thiamine is often greater.

Vitamin B₆ (pyridoxine) is known for its role in amino acid metabolism. Due to pyridoxine's role in the metabolism of protein and amino acids, its requirement by the body increases as protein intake is increased. Pyridoxine is also involved of the conversion of linoleic acid to arachidonic acid, glycogenolysis (glycogen breakdown) energy production, and synthesis of red blood cells.

Vitamin B₁₂ is regarded as the primary energy vitamin. Vitamin B₁₂ functions in the body in new cell growth nerve tissue development, folate metabolism, and DNA synthesis and energy production. It is necessary for the synthesis of red blood cells and, therefore, for the prevention of anemia.

Folate functions metabolically as a coenzyme that transports carbon molecules from one compound to another in amino acid metabolism and nucleic acid synthesis. Folate affects the tissues that grow rapidly, such as skin, the lining of the gastrointestinal tract, the bone marrow, and regenerating muscle tissue.

Osmotically Balanced Electrolytes, Sodium, Potassium and Magnesium

Osmotically Balanced is defined herein as a product that enhances the body's cellular ability to absorb, efficiently and effectively, the ingredients and nutrients for optimum utilization. Osmotically balanced electrolytes facilitate uptake of water and nutrients while minimizing digestive distress. Sodium is the body's main extra-cellular cation. Sodium helps regulate the body's volume of extra-cellular fluids, particularly the blood. It also helps regulate pressure of these fluids, aids the active transport of nutrients across cell membranes, and assists the uptake of some nutrients in the intestines. In addition, it functions in muscle contraction and nerve impulse transmission. Significant amounts of sodium are lost through perspiration and must be replenished.

Potassium functions in the body primarily as an intercellular fluid cation. It helps maintain fluid balance and functions in nerve transmission muscle contraction and glycogen formation.

Magnesium plays many metabolic and structural roles. It helps form bone and teeth, functions in muscle tissue development and the proper function of the nervous system, as well as a cofactor for activating many enzymes. It assists in calcium and potassium uptake and glycolysis, as well as many other metabolic processes. It aids the muscle to relax preventing muscle spasms, tremors and convulsions. It is a critical element in the treatment of chronic fatigue, which involves muscle aches and pains.

Calcium, Zinc and Chromium

In addition to calcium's role as the primary nutrient needed for bone formation and maintenance, calcium also plays essential roles in nerve conduction, nerve impulse transmission, heart rate, muscle contraction, cell-membrane permeability, and blood clotting. It also functions as an enzyme cofactor.

Zinc plays many important metabolic roles in the body. It is part of various metallo-enzymes (mineral-containing enzymes) that function in growth, testosterone production, DNA synthesis, cell replication and prostate gland functioning.

Chromium's role is as a potentiator of insulin; that is, it helps insulin do its job. It is an important cofactor for energy production and tissue growth and repair. It performs this function by helping glucose and amino acids circulating in the blood stream to be taken up by the cells at a higher rate.

Amino Acids: Straight-Chain Amino Acids L-Glutamine and Glycine, and Essential Branched-Chain Amino Acids L-Leucine, L-Valine and L-Isoleucine

L-glutamine is essential for the proper functioning of the brain. It is an energy source in the brain and a mediator of glutamic acid and GABA activity. L-glutamine is also vital to immune system functioning and is required for cellular replication in the immune system. However, the majority of L-glutamine is made in the muscles.

Glycine is an important precursor of many substances, including protein, DNA, phospholipids, collagen, and creatine. It is also a precursor in the release of energy. It is necessary for the proper functioning of the central nervous system and is an inhibitory neurotransmitter.

L-leucine is an essential amino acid found in proteins, like the other branched-chain amino acids (BCAAs), important in energy production during exercise. According to estimates, up to 90 percent of dietary L-leucine may be used for energy in existing muscles. This makes L-leucine a very limiting amino acid if supplemental amounts are not taken to compensate for the loss. L-leucine has been shown to help spare muscle tissue, maintain nitrogen balance, and promote muscle growth and healing.

L-valine is involved in tissue repair, nitrogen balance, and muscle metabolism. It regulates how the body uses protein and plays a unique role in protein metabolism in muscles. Intense physical exercise produces a rapid excretion of nitrogen, which causes a decrease in muscle protein synthesis. L-valine limits this decrease.

L-Isoleucine, as the other BCAAs, is an integral part of muscle tissue and may be used for energy by exercising muscles. L-isoleucine is found in proteins and is needed for the formation of hemoglobin. It is involved in the regulation of blood sugar and is metabolized for energy in muscle tissue during exercise. Intense physical exercise produces a rapid excretion of nitrogen, which causes a decrease in muscle protein synthesis. L-isoleucine limits this decrease.

Botanicals

Green Tea Leaf Extract has been renowned as the herbal healer for over 4000 years. It is one of the best sources of polyphenols—naturally occurring plant chemicals that have amazing antioxidant properties. Since athletes, through exertion and exercise, build up excess free radicals it is critical to supplement their diets with the strongest antioxidants available.

Grape Seed Extract is one of the most effective dietary antioxidants. As the body builds up free radicals and oxidative stress, certain enzymatic free radical scavenging and salvage systems are simply not capable of handling the onslaught. This cumulative effect of free radical damage over time may diminish the cell's ability to make these repairs. Grape Seed Extract has proven effective in reversing oxidative stress and free radical damage.

Ferulic Acid is a substance extracted from rice bran oil that reportedly promotes a variety of metabolic effects. These metabolic effects include increased endorphin release, antioxidant activity, lipotropic action, stress reduction, growth hormone stimulation, increased growth, and improved recovery.

Siberian Ginseng belongs to a different botanical genus than Chinese, Korean and American ginseng, but properties and uses are similar. It is generally accepted as a strong herbal stimulant of both mental and physical energy. The only ginseng that contain eleutherosides, which when combined with amino acids stimulate the generation of energy and building of lean muscle mass.

Korean Ginseng belongs to same botanical genus than Siberian ginseng, but its properties and uses are similar. Also known as Asiatic Ginseng the wonder of the world is traditionally used for the production of energy. It is generally accepted as a strong herbal stimulant of both mental and physical energy.

Other Probiotics: Amino Sugars and Ornithine α-Ketoglutarate

Glucosamine, an amino sugar, has properties that benefit all types of connective tissue. Connective tissue contains two main components. The chief component is collagen, which is the most common protein in the body, making up one-third of the body's total protein volume. The other component is proteoglycan, which forms the framework for collagenous tissue and also acts as a lubricant in joints. Glucosamine is one of the few known ingredients that aid in the synthesis of proteoglycan.

Ornithine α-ketoglutarate (OKG) is popular due to the marvelous features it offers the athlete. OKG has been used to treat the victims of burns, trauma, or severe malnutrition and aid post-surgical healing. It acts as an ammonia scavenger, increases the L-glutamine pool in muscle tissue, thereby reducing catabolism, elevates the growth hormone level, increases protein synthesis, increases insulin secretion, and aids L-glutamine synthesis.

### DESCRIPTIVE RELATIONSHIPS OF THE 29 INGREDIENTS USED IN THE FORMULATION OF THE PRESENT INVENTION

Note: “Active Ingredients” are those ingredients that have a daily requirement set by the Federal Drug Administration. The terminology is not intended to imply that “Inactive” ingredients lack functionality.

Active Ingredients:

Vitamin A (as retinyl acetate)

Quantity—5,000 IU (mg)

Value/range—100% daily value

Importance—Vitamin A plays many roles in the body. It is essential for maintenance of vision and proper cellular growth and development. It is necessary for reproduction, since it is involved in the proper function of the reproductive organs. Vitamin A is needed for the proper maintenance and integrity of the immune system; for the formation and maintenance of healthy skin, hair, and mucous membranes; as well as for bone growth and tooth development. It also has been implied that it has or performs some anticancer functions. This suggests that maintaining a proper intake of the carotenoids may help reduce the risk of lung cancer.

Performance Parameters—Scientists speculate that this anticancer activity is due partially to Vitamin A's role in promoting the development of normal epithelial cells. Epithelial cells compose such structures as the linings of the lungs and digestive system. In addition to all of Vitamin A's functions, it is an antioxidant, able to quench free radicals, particular singlet oxygen. This helps reduce cellular, molecular, and tissue damage by free radicals, whose numbers are greatly elevated by exercise and increased oxygen uptake.

Alternative Ingredient—None known. In this formulation this ingredient is critical because Vitamin A is not a water soluble Vitamin. This form of Vitamin A (ingredient) is more readily soluble in water, which is critical to the taste and clear appearance of the drink. Other forms of Vitamin A can leave an oily residue and or result in a cloudy solution. They can also discolor the skin. It also remains in solution and will not settle when chilled or left in solution for long periods of time while maintaining its potency.

Limitations/Undesired Characteristics—None known for this dosage.

Vitamin C (as ascorbic acid)

Quantity—180 mg

Value/range—300% daily value

Importance—Vitamin C (ascorbic acid) is primarily a water-soluble antioxidant that cannot be synthesized by the human body. Neither is it stored in the body in any significant amount. Therefore, it is an essential vitamin. In addition, a slightly altered form of ascorbic acid, called dehydroascorbic acid, is present in the diet. This oxidized form also displays some Vitamin C activity. It has multiple functions as a cofactor and coenzyme. It is involved in the formation and maintenance of collagen, a protein that is an important component of skin, ligaments, and bones. It helps heal wounds, may help fight infections, and promotes healthy capillaries, gums and teeth. It prevents the oxidation of folate, aids in the intestinal absorption of iron, and assists in the metabolism of tyrosine and phenylalanine. It protects cells from free radical damage and blocks the production of nitrosamines, organic compounds present in various foods and found to produce cancer in laboratory animals.

Performance Parameters—Today, many people tout it as a general cure-all. For athletes, studies have indicated that Vitamin C plays a part in increasing muscular strength, reducing the blood-lactate-level, and sparing glycogen.

Alternative Ingredient—None known. It is the most effective form of Vitamin C. In this formulation this ingredient is needed because of it is readily soluble in water, which is critical to the taste and clear appearance of the drink. It also remains in solution and will not settle when chilled or left in solution for long periods of time while maintaining its potency.

Limitations/Undesired Characteristics—None known for this dosage.

Vitamin E (as dl-alpha tocopheryl acetate)

Quantity—60 IU (mg)

Value/range—200% daily value

Importance—Vitamin E serves a host of functions. It assists in red blood cell formation, acts as an antioxidant, and aids in the regulation of prostanoid synthesis. Prostanoids are compounds that are important in the reproductive process, and blood-platelet aggregation involved in control of bleeding. Vitamin E protects cell membranes against oxidation, prevents blood clots, retards oxidation of the other fat-soluble vitamins, helps cells respire, and treats or prevents vitamin deficiency. Of interest to athletes, Vitamin E has also been shown to lower the blood-lactate level, decrease the formation of certain waste products during exercise, reduce oxidative cellular damage, maintain muscle tissue, and possibly help testosterone production.

Performance Parameters—In studies intake of Vitamin E have been found to benefit athletes by improving energy production, reducing cellular damage, and stabilizing membranes.

Alternative Ingredient—None known. In this formulation this ingredient is critical because Vitamin E is not a water-soluble vitamin. This ingredient is more readily soluble in water, which is especially critical to the taste and clear appearance of the drink. It also remains in solution and has minimal settling properties when chilled or left in solution for long periods of time. It also maintains its potency when left in solution for long periods of time.

Limitations/Undesired Characteristics—None known for this dosage.

Vitamin B₁ (as thiamine HCl)

Quantity—10 mg

Value/range—666%

Importance—Thiamine is used in the body as a coenzyme that aid in carbohydrate metabolism. Since athletes tend to eat more carbohydrates than non-athletes do, their requirement for thiamine is greater.

Performance Parameters—Thiamine also functions in the production of ribose, which is needed for the synthesis of RNA and DNA and for appetite stimulation. It also helps maintain the integrity of nervous system functioning, which is extremely beneficial in athletes. Endurance athletes have reported improvements in performance from ingesting increased amounts of thiamine.

Alternative Ingredients—None known. It's function and maintenance of the nervous system can not be duplicated. In this formulation this ingredient is also critical because it is readily soluble in water which is critical to the taste and clear appearance of the drink. It also remains in solution and has minimal settling properties when chilled or left in solution for long periods of time. It also maintains its potency when left in solution for long periods of time.

Limitations/Undesired Characteristics—None known for this dosage.

Vitamin B₆ (as pyridoxine HCL)

Quantity—8 mg

Value/rating—400%

Importance—Pyridoxine is known for its role in amino acid metabolism. It functions primarily in transamination reactions, processes in which an amino group is transferred from an amino acid to another molecule, usually to produce a different amino acid. Due to pyridoxine's role in the metabolism of protein and amino acids, its requirement by the body increases as protein intake is increased. Pyridoxine is also involved of the conversion of linoleic acid to arachidonic acid, glycogenolysis (glycogen breakdown) energy production, and synthesis of red blood cells.

Performance Parameters—Studies using pyridoxine with athletes have had results similar to those using niacin with athletes due to pyridoxine's tendency to increase the bodies utilization of its glycogen stores and to decrease its use of fatty acids. Endurance athletes should therefore avoid taking megadoses of pyridoxine. However, as with niacin, there is some evidence that consuming high doses of pyridoxine before short-term anaerobic exercise may enhance performance. Again, this is probably because anaerobic athletes, such as weight lifters and sprinters get more of their energy from stored glycogen, and faster glycogen release may result in greater power output. Athletes going through the glycogen-depletion phase of a carbohydrate loading program can increase depletion of their glycogen stores with as little as 8 milligrams of pyridoxine a day. Pyridoxine has also reportedly increased the exercise-induced rise in growth hormone, which is another potential benefit for power athletes.

Alternative Ingredients—None known. There are no other vitamins that perform the specific interactive functions in metabolism. It also reacts with the maltodextrin compounds in a unique manner so as to generate instant energy. In this formulation this ingredient is also critical because it is readily soluble in water which is critical to the taste and clear appearance of the drink. It also remains in solution and has minimal settling properties when chilled or left in solution for long periods of time. It maintains its potency when left in solution for long periods of time.

Limitations/Undesired Characteristics—None known for this dosage.

Folate (folic acid)

Quantity—400 μg

Value/range—100% daily value

Importance—Folate is a group of related compounds that have nutritional properties similar to other B vitamin complexes.

Performance Parameters—These compounds function metabolically as coenzymes that transport carbon molecules from one compound to another in amino acid metabolism and nucleic acid synthesis. In this way folate is very important as a cofactor in DNA and RNA formation, protein synthesis, and cellular replication. Folate also stimulates the formation of red blood cells and Vitamin B₁₂. In particular, Folate affects the tissues that grow rapidly, such as skin, the lining of the gastrointestinal tract, the bone marrow, and regenerating muscle tissue. Athletes will benefit from higher than average folate intake as it produces a faster recovery and growth rate.

Alternative Ingredients—None known. There are no other vitamins that perform these specific interactive functions in amino acid metabolism. Its reaction as a cofactor in DNA and RNA make it particularly important in the generation of energy and the stimulation of the healing of soft tissue. In this formulation this ingredient is also critical because it is readily soluble in water which is critical to the taste and clear appearance of the drink. It also remains in solution and has minimal settling properties when chilled or left in ingredient is critical because it is readily soluble in water, which is critical to the taste, and clear appearance. It maintains its potency when remaining in solution for long periods of time.

Limitations/Undesired Characteristics—None known for this dosage.

Vitamin B₁₂ (as cyanocobalamin) p1 Quantity—9 μg

Value/range—150% daily value

Importance—Vitamin B₁₂ is only one part of the nutrition picture, but it is a very important part, playing an essential role in maintaining athletic performance. It is regarded as the primary energy vitamin. In fact, a common practice among athletes is to get Vitamin B₁₂ shots during the season. Vitamin B₁₂, also known as cobalamin, is really a group of cobalt-containing compounds that display Vitamin B₁₂ activity. Vitamin B₁₂ functions in the body in new cell growth nerve tissue development, folate metabolism, DNA synthesis and energy production. It is necessary for the synthesis of red blood cells and, therefore, for the prevention of anemia.

Performance Parameters—Studies conducted in the 1980s on non-athletes experiencing tiredness are credited with initiating the current practices of taking mega-doses of Vitamin B₁₂ and injecting B₁₂ that are so widespread among athletes. These studies use only Vitamin B₁₂ injections, though, the benefit of consuming mega-doses of B₁₂ have not yet been substantiated by research. Furthermore, various studies examining the strength and endurance effects of B₁₂ have not showed the vitamin to provide any immediate benefits. To date, Vitamin B₁₂'s primary function is metabolic. Researchers are currently focusing their attention on a coenzyme form of B₁₂ called cobamamide. Cobamamide is being touted as an anabolic form of B₁₂ that is comparable to anabolic steroids. According to athletes, however, cobamamide raises the perceived energy level and does increase appetite. Therefore, the use of coenzyme B₁₂ along with Vitamin B₁₂ is recommended for athletes.

Alternative Ingredient—None known. Its unique properties in the production of energy and increasing stamina and endurance con not be replaced. In this formulation this ingredient is also critical because it is readily soluble in water which is critical to the taste and clear appearance of the drink. It also remains in solution and has minimal settling properties when chilled or left in solution for long periods of time. It also maintains its potency when left in solution for long periods of time.

Limitations/Undesired Characteristics—None known for this dosage.

Calcium (as calcium carbonate)

Quantity—200 mg

Value/range—20% daily value

Importance—The average adult body contains approximately 1,200 grams of Calcium, 99 percent of, which is located in the skeleton. Calcium is found in the skeleton primarily as calcium phosphate. Calcium also occurs in the body in ionic form and as calcium carbonate. In addition to its role as the primary nutrient needed for bone formation and maintenance, Calcium also plays essential roles in nerve conduction, nerve impulse transmission, heart rate, muscle contraction, cell-membrane permeability, and blood clotting. It also functions as a coenzyme. Recently, calcium level was found to be correlated to the control of blood pressure in some individuals. In bone formation and maintenance, a positive calcium balance, that is, more calcium absorbed than excreted is required for proper mineralization of bone. This positive balance must be maintained during the growth years and throughout adulthood. Until recently, most medical authorities believed that after the age of thirty, building more bone tissue became impossible.

Performance Parameters—Research has proven what many sports-fitness scientists already knew—exercise and the proper dietary intake of Calcium result in increased bone density in adults of all ages. For athletes these findings mean that an adequate calcium level must be maintained year round, from childhood through the senior years.

Alternative Ingredient—None. Calcium carbonate contains one of the highest amounts of elemental calcium per milligram of all the calcium salts. It is also the most readily absorbable of the calcium salts. A critical element in the maintenance of proper hydration and in the prevention of dehydration when used in the correct relationship to the other electrolytic elements.

Limitations/Undesired Characteristics—None known.

Magnesium—(as magnesium oxide)

Quantity—120 mg

Value/range—30% daily value

Importance—Most of the magnesium that is present in the body is located in the bones, muscles and soft tissues. Altogether, the average adult body contains about 24 grams of magnesium. Magnesium plays many metabolic and structural roles. It helps form bone and teeth, functions in muscle tissue development and the proper function of the nervous system, as well as activating enzymes. It assists calcium and potassium uptake, glycolysis, as well as many other metabolic processes. It aids the muscle to relax preventing muscle spasms, tremors and convulsions. It is a critical element in the treatment of chronic fatigue, which involves muscle aches and pains.

Performance Parameters—Athletes should note that several studies have shown that supplementing the diet with moderate amounts of magnesium improves several performance factors including endurance and strength. Researchers have also observed that athletes that increase their physical activity tend to deplete their magnesium stores, this especially true in endurance athletes.

Alternate Ingredient—None. In this formulation this ingredient is critical because of its solubility which is critical to the taste, color and clear appearance of the drink. It also remains in solution and has no settling properties when chilled or left in solution for long periods of time. It also maintains its potency when left in solution for long periods of time, which is critical to the overall effectiveness of the macronutrient modulation and interaction with other nutrients. A critical element in the maintenance of proper hydration and in the prevention of dehydration when used in the correct relationship to the other elements.

Limitations/Undesirable Characteristics—None known for this dosage.

Zinc—(as zinc oxide)

Quantity—10 mg

Value/range—66% daily value

Importance—In athletic circles, zinc has acquired the reputation of being one of the primary healing nutrients and a major contributor to male fertility. Zinc plays many important metabolic roles in the body. It is part of various metallo-enzymes (mineral-containing enzymes) that function in growth, testosterone production, DNA synthesis, cell replication, fertility, reproduction, and prostate gland functioning. It occurs in ionic form in cells, assists in the synthesis of molecules, and serves as a component of enzymes. For athletes, maintaining proper zinc intake is vital, especially for the growth and repair of muscle tissue to meet the demands of training.

Performance Parameters—Very few studies have been conducted to examine the effects of zinc supplementation on performance, although one study did show that the effect is increased muscle endurance. Further research will indicated whether or not there are additional benefits for performance.

Alternative Ingredient—None. This is of the forms of elemental zinc which are referred to as Chelated Zinc. It is a relatively inexpensive form of zinc and is well tolerated by the stomach and system. In this formula this ingredient is critical because of its solubility which is critical to the color and clear appearance of the drink. It is also one of the easiest elemental form to mask the taste. It also remains in solution and has no settling properties when chilled or left in solution for long periods of time which is critical for heavy element.

Limitations/Undesirable Characteristics—After taste. No others known for this dosage.

Chromium (as chromium dinicotinate glycinate)

Quantity—100 μg

Value/range—83% of daily value

Importance—Chromium's major role is as a potentiator of insulin, that is, it helps insulin do its job. Chromium is an important cofactor for energy production and tissue growth and repair. For the athlete, adequate Chromium intake is essential.

Performance Parameters—Chromium's major role is as a potentiator of insulin, that is, it helps insulin do its job. Chromium also assists in formation, structural maintenance, and metabolism of the nucleic acids. It helps fatty acid and cholesterol formation in the liver and according to some studies, may help lower cholesterol. Furthermore, chromium-deficient diets have been linked to higher incidents of diabetes and heart disease. It is chromium's role as an insulin potentiator that has brought it the recent media attention. Early researchers found that chromium helps lower the blood-sugar level. Because of this, chromium has become known as the glucose tolerance factor. Chromium performs this function by helping glucose and amino acids circulating in the blood stream to be taken up by the cells at a higher rate. This does not necessarily mean that the level of insulin is increased, nor does it mean that chromium has a direct effect on muscle building, the way testosterone does, or on fat loss, the way growth hormone does. It just means that increasing the dietary chromium level should help improve insulin functioning. The cells use glucose and amino acids for energy and growth. If insulin functioning is not 100 percent efficient, the glucose and amino acids in the blood stream circulate back to the liver and are possibly converted to fat. Insulin malfunctioning can also lead to the development of diabetes, heart disease, or other metabolic disorders. Chromium's role in insulin functioning does not make it an anabolic agent either. Several studies have shown that taking supplemental chromium in association with a training program and good diet has helped increase the rate of muscle gain as well as the rate of fat loss.

Alternative Ingredient—None. Chromium's function in the rapid absorption of glucose and amino acids circulating in the blood stream and in formation, structural maintenance, and metabolism of the nucleic acids make it necessary. In this formulation this ingredient is also critical because of its solubility which is critical to the taste, color and clear appearance of the drink. It also remains in solution and has no settling properties when chilled or left in solution for long periods of time.

Limitations/Undesirable Characteristics—None known for this dosage.

Sodium—(as sodium chloride and sodium citrate)

Quantity—195 mg

Value/range—8% daily value

Importance—While potassium is the body's main intercellular cation, sodium is the body's main extracellular cation. Sodium helps regulate the body's volume of extra-cellular fluids, particularly the blood. It also helps regulate pressure of these fluids, aids the active transport of nutrients across cell membranes, and assists the uptake of some nutrients in the intestines. In addition, it functions in muscle contraction and nerve impulse transmission.

Performance Parameters—Studies support the adequate intake of sodium for the maintenance of overall health and athletic performance. A critical element in the maintenance of proper hydration and in the prevention of dehydration if used in the correct relationship to the other elements.

Alternative Ingredient—None. In this formulation these ingredients are essential because of their solubility which is critical to the taste, color and clear appearance of the drink. It also remains in solution and has no settling properties when chilled or left in solution for long periods of time. It also maintains its potency when left in solution for long periods of time, which is critical to the overall effectiveness of the macronutrient modulation and interaction with other nutrients. A critical element in the maintenance of proper hydration and in the prevention of dehydration when used in the correct relationship to the other elements.

Why sodium chloride and citrate instead of phosphate?

1. The main reason for the choice of chloride and citrate was the taste and color properties for our drink.

2. They are very conducive to drinks with a fruit flavor.

3. Sodium phosphate is one of the most active forms and is used in cola type drinks; it is very conducive to cola taste and color and not good for fruit flavor and color drinks.

Limitations/Undesirable Characteristics—None known for this dosage.

Potassium—(as potassium bicarbonate & dipotassium phosphate)

Quantity—285 mg

Value/range—8% daily value

Importance—Potassium functions in the body primarily as an intercellular fluid cation. A cation is a positively charged ion. Intercellular fluid is any fluid that is found within a cell. As an intercellular cation potassium is essential to all living cells. It helps maintain fluid balance and functions in nerve transmission muscle contraction and glycogen formation.

Performance Parameters—Studies support the adequate intake of potassium for maintenance and overall health and athletic performance. A critical element in the maintenance of proper hydration and in the prevention of dehydration if used in the correct relationship to the other elements.

Alternative Ingredient—None. In this formulation these ingredients are essential because of their solubility which is critical to the color and clear appearance of the drink. It also remains in solution and has no settling properties when chilled or left in solution for long periods of time. It also maintains its potency when left in solution for long periods of time, which is critical to the overall effectiveness of the macronutrient modulation and interaction with other nutrients. A critical element in the maintenance of proper hydration and in the prevention of dehydration when used in the correct relationship to the other elements.

Limitations/Undesirable Characteristics—None known for this dosage.

Inactive Ingredients:

Carbohydrates (maltodextrin 55%, dextrose 30%, sucrose 15%)

Quantity—60 g

Value/range—20% daily value (for 2,000 calorie diet)

Importance—Carbohydrates are the major source of energy for physical activities.

Performance Parameters—Complex carbohydrates are the best source because they most effectively refill the glycogen stores in the muscles and liver. In addition, they elevate and maintain the blood sugar to a level sufficient for long sessions of intensive training. Research has shown that athletes must make sure that they consume adequate amounts of both complex carbohydrates and protein. If the athlete's energy stores become drastically depleted or they experience lactic-acid build up, they will suffer temporary muscle fatigue. If the glycogen stores are not replenished before the next work out or game, their body will begin breaking down muscle tissue for the protein it needs to generate energy. The use of polysaccharides (maltodextrins) as well as simple sugars regulates the rise in blood sugar following ingestion and allows for sustained even blood sugar levels without a sugar “crash”.

Alternative Ingredient—None. In this formulation this ingredient is used as a sweetener as well as the generating source of energy. This form is used in electrolyte or rehydration formulations because of its effectiveness. This ingredient is readily soluble in water, which is critical to the clear appearance of the drink. This specific mixture of carbohydrates, because of its gravity remains in solution and will not settle at room temperature, when left in solution for long periods of time or when chilled.

Limitations/Undesired Characteristics—Can have some negative impact on people with diabetes.

L-Glutamine

Quantity—700 mg

Value/range—None

Importance—L-glutamine is formed from glutamic acid by the addition of ammonia catalyzed by vitamin B₆. L-glutamine is a neurotransmitter in the brain, where it can be converted back to glutamic acid. It is essential for the proper functioning of the brain. It is an energy source in the brain and a mediator of glutamic acid and GABA activity. L-glutamine is also vital to immune system functioning. New studies show that L-glutamine is required for cellular replication in the immune system. However, the majority of L-glutamine is made in the muscles.

Performance Parameters—Use of supplemental free-form L-glutamine by athletes is known to produce a strong anticatabolic effect, which neutralizes the cortisol that accompanies strenuous exercise. Cortisol is a steroid hormone and highly catabolic. L-glutamine's anticatabolic action allows more efficient anabolism. L-glutamine is also active in recovery and healing. Is active in alleviating fatigue, improving brain function and as a mood elevator.

Alternative Ingredient—None available.

Limitations/Undesirable characteristics—None known.

Glycine

Quantity—175 mg

Value/range—None

Importance—Glycine is an important precursor of many substances, including protein, DNA, phospholipids, collagen, and creatine. It is also a precursor in the release of energy. Glycine is used by the liver in the elimination of phenols, which are toxic, and in the formation of bile salts. It is necessary for the proper functioning of the central nervous system and is an inhibitory neurotransmitter. Too much supplemental Glycine can displace glucose in the metabolic energy chain and cause fatigue, but just enough can help produce more energy.

Performance Parameters—Some studies have also noted that glycine ingestion causes an increase in strength, possibly due in part to its elevation of the growth hormone levels. Supplemental glycine has additionally been shown to increase the creatine level the major source of muscle. Glycine should be part of all full-spectrum amino acid supplements.

Alternative Ingredient—None available.

Limitations/Undesirable Characteristics—None known for this dosage.

L-Valine

Quantity—175 mg

Value/range—None

Importance—L-Valine is an essential amino acid and a member of the branched-chain amino acids (BCAAs). Like other BCAAs, it is an integral part of muscle tissue and may be used for energy by exercising muscles. It is involved in tissue repair, nitrogen balance, and muscle metabolism. BCAAs regulate how the body uses protein and play a unique role in protein metabolism in muscles. They are oxidized in peripheral muscles. They are the principal source of calories for muscle tissue. Intense physical exercise produces a rapid excretion of nitrogen, which causes a decrease in muscle protein synthesis. L-valine supplementation limits this decrease.

Performance Parameters—Studies have shown that L-valine balances insulin secretions and directly affects muscle and body weight changes and promotes lean muscle distribution. L-valine has been shown to reduce appetite while preserving basic protein storage in the body. It also has been demonstrated to be available to muscles in one hour.

Alternative Ingredient—None available.

Limitations/Undesirable Characteristics—None known.

L-Isoleucine

Quantity—175 mg

Value/range—None

Importance—L-Isoleucine is an essential amino acid found in proteins that is, one of the branched-chain amino acids (BCAAs). Like the other BCAAs, it is an integral part of muscle tissue and may be used for energy by exercising muscles. L-isoleucine is found in proteins and is needed for the formation of hemoglobin. It is involved in the regulation of blood sugar and is metabolized for energy in muscle tissue during exercise. Supplemental intake of L-isoleucine, along with the other BCAAs, has been shown to help spare muscle tissue, maintain nitrogen balance, and promote muscle growth and healing. BCCAs regulate how the body uses protein and play a unique role in protein metabolism in muscles. They are oxidized in peripheral muscles. They are the principal source of calories for muscle tissue. Intense physical exercise produces a rapid excretion of nitrogen, which causes a decrease in muscle protein synthesis. L-isoleucine supplementation limits this decrease.

Performance Parameters—Studies have shown that L-isoleucine balances insulin secretions and directly affects muscle and body weight changes and promotes lean muscle distribution. L-isoleucine has been shown to reduce appetite while preserving basic protein storage in the body. It also has been shown to be available to muscles in one hour.

Alternative Ingredient—None available.

Limitations/Undesirable Characteristics—None known.

L-Leucine

Quantity—350 mg

Value/range—None

Importance—L-leucine is an essential amino acid found in proteins that is, like the other branched-chain amino acids (BCAAs), important in energy production during exercise. For many years, the three BCAAs were assumed to contribute equally to energy. Recent studies, however, have shown that both exercising and resting muscle tissue use far more L-leucine for energy than either of the other two BCAAs. According to estimates, up to 90 percent of dietary L-leucine may be used for energy in existing muscles. This makes L-leucine a very limiting amino acid if supplemental amounts are not taken to compensate for the loss. Supplemental intake of L-leucine, along with the other BCAAs, has been shown to help spare muscle tissue, maintain nitrogen balance, and promote muscle growth and healing. They regulate how the body uses protein and play a unique role in protein metabolism in muscles. They are oxidized in peripheral muscles. They are the principal source of calories for muscle tissue. Intense physical exercise produces a rapid excretion of nitrogen, which causes a decrease in muscle protein synthesis. Leucine limits this decrease.

Performance Parameters—Studies have shown that it balances insulin secretions and directly affects muscle and body weight changes and promotes lean muscle distribution. L-leucine has been shown to reduce appetite while preserving basic protein storage in the body. It also has been shown to be available to muscles in one hour.

Alternative Ingredient—None available.

Limitations/Undesirable Characteristics—None known.

Green Tea Leaf Extract (36% total polyphenols)

Quantity—50 mg

Value/rating—None

Importance—For over 4000 years Green Tea has been renowned as the herbal healer. It is one of the best sources of polyphenols or naturally occurring plant chemicals that have amazing antioxidant potential. Since athletes, through exertion and exercise, build up excess free radicals it is critical to supplement their diets with the strongest antioxidants available, which enables the body to heal and repair itself.

Performance Parameters—Modern scientific research has now confirmed that it offers real benefits in the prevention and contributes to the cure of a variety of diseases. Studies have also proven that it boosts the immune system and is one of the best sources of polyphenols or naturally occurring plant chemicals, that have amazing antioxidant potential. Green Tea has been proven to strengthen the heart, capillary walls, and lower blood pressure. As an antioxidant it is one of the most active, and researcher have said that the antioxidant activity of the four polyphenols in Green Tea: epicatchin, epicatechin gallate, epigallocatechin, and epigallocatechin gallate or (EGCG) seems to be superior to that of better known antioxidants Vitamins C and E. Research at the University of Southern California is dedicated to understanding free radicals, the damage they cause, and the ways in which to combat them or prevent their formation. According to this research, the body built up repair mechanisms—certain enzymatic free radical scavenging and salvage systems—are not capable of handling the onslaught. This cumulative effect of free radical damage over time may diminish the cell's ability to make these repairs.

Alternative Ingredients—None available. The strongest and most effective herbal antioxidant known. Its healing properties have been recognized for over 4,000 years. In this formula any other plant chemical can not duplicate the overall effects on the immune system and the circulatory system. It also remains in solution and has no settling properties when chilled or left in solution for long periods of time.

Limitations/Undesirable Characteristics—None known.

Grape Seed Extract (50% proanthocyanidins)

Quantity—25 mg

Value/rating—None

Importance—Modern scientific research has now confirmed that Grape Seed Extract offers distinct benefits in the prevention of a variety of diseases. As an antioxidant it is one of the most effective. Without adequate antioxidants body repair mechanisms are not able to handle the onset of free radicals and oxidative stress caused by vigorous physical activity. The cumulative effect of free radical damage over time may diminish the cell's ability to make repairs and to grow. Since athletes, through exertion and exercise, build up excess free radicals it is critical to supplement their diets with the strongest antioxidants available.

Performance Parameters—Studies have also proven that it boosts the immune system and is another of the best sources of polyphenols or naturally occurring plant chemicals, that have amazing antioxidant potential. Grape Seed Extract has long been used in the treatment of diseases relating to the arteries, veins, cardiovascular system, high blood pressure, water retention as well as damage to collagen, a factor in arthritis. As an antioxidant it is one of the most effective. Research has demonstrated the effectiveness of the active phytochemical compounds, or flavonoids, found in this ingredient. Research at the University of Southern California is dedicated to understanding free radicals, the damage they cause, and ways in which to combat them or prevent their formation. According to this research, the body built up repair mechanisms—certain enzymatic free radical scavenging and salvage systems—are not capable of handling the onslaught.

Alternative Ingredients—None available. Because of the unique regeneration and antioxidant properties that are associated with this herbal compound it is not possible to find a replacement ingredient that will be as effective in this formulation. It's use in the treatment of diseases relating to the arteries, veins, cardiovascular system, high blood pressure, water retention as well as damage to collagen, a factor in arthritis make it the herb of choice. It also remains in solution and has no settling properties when chilled or left in solution for long periods of time.

Limitations/Undesirable Characteristics—None known.

Ferulic Acid (FRAC)

Quantity—50 mg

Value/rating—None

Importance—Ferulic Acid, is a substance extracted from rice bran oil that reportedly promotes a variety of metabolic effects. These metabolic effects include increased endorphin release, antioxidant activity, lipotropic action, stress reduction, growth hormone stimulation, increased growth, and improved recovery.

Performance Parameters—The metabolic effects of Ferulic Acid include increased strength, improved recovery, reduced muscle soreness, reduced sensation of fatigue, and decreased catabolic effects of cortisol. While research is sparse, athletes' report beneficial results from the supplemental use of Ferulic Acid. Ferulic Acid is very bioavailable, and is reported to have no toxic side effects. For optimum results, take supplemental Ferulic Acid before workouts on training days and in the morning on non-training days.

Alternative Ingredients—None known. Ferulic Acid is about 30 times more bioavailable than Gamma Oryzanol, the nearest replacement ingredient, and is reported to have no toxic side effects. In this formulation this ingredient is also critical because of its solubility which is critical to the taste, color and clear appearance of the drink. It also remains in solution and has no settling properties when chilled or left in solution for long periods of time.

Limitations/Undesirable Characteristics—None known.

Siberian Ginseng Root Extract (0.8% eleutherosides B & E)

Quantity—75 mg

Value/rating—None

Importance—Siberian Ginseng belongs to a different botanical genus than Chinese and American ginseng, but properties and uses are similar. It is generally accepted as a strong herbal stimulant of both mental and physical energy. This is the only Ginseng that contain eleutherosides, which when combined with amino acids stimulate the generation of energy and building of lean muscle mass.

Performance Parameters—Ginseng is traditionally used for energy. It increases the energy level, eases stress, and supports the immune system. Oriental cultures have been using ginseng for 5,000 years and still revere it as a cure all. It is a mild laxative and helps the body pass poisons more rapidly. It aids the body in the rapid assimilation of vitamins and minerals acting as an Endocrine-Gland stimulant.

Alternative Ingredient—None available. This ingredient because of its solubility in water and taste enhances the color and taste of the drink. It also remains in solution and has no settling properties when chilled or left in solution for long periods of time.

Limitations/Undesirable Characteristics—Should not be used by anyone with hypoglycemia, high blood pressure or heart disease.

Korean Ginseng Extract (5% ginsenosides)

Quantity—150 mg

Value/rating—None

Importance—Korean Ginseng belongs to a different botanical genus than Siberian Ginseng, but properties and uses are similar. Also known as Asiatic Ginseng, the wonder of the world, it is traditionally used for the production of energy. It is generally accepted as a strong herbal stimulant of both mental and physical energy.

Performance Parameters—Ginseng is traditionally used for energy. It increases the energy level, eases stress, and supports the immune system. Oriental cultures have been using ginseng for 5,000 years and still revere it as a cure all. It is a mild laxative and helps the body pass poisons more rapidly. It aids the body in the rapid assimilation of vitamins and minerals acting as an Endocrine-Gland stimulant. It also is known to counteract the effects of aging, improves sleep, and promotes recovery and tissue repair. Compliments and multiplies the impact and effects of Siberian Ginseng.

Alternative Ingredients—None available. This ingredient because of its solubility in water and taste enhances the color and taste of the drink. It also remains in solution and has no settling properties when chilled or left in solution for long periods of time.

Limitations/Undesirable Characteristics—Should not be used by anyone with hypoglycemia, high blood pressure or heart disease.

Glucosamine HCL (as glucosamine hydrochloride)

Quantity—300 mg

Value/rating—None

Importance—Glucosamine, an amino sugar, as a supplement is widely heralded as an effective treatment for arthritis. It has properties that also benefit all types of connective tissue. These different types of connective tissue make up the tendons, ligaments, intravertebral discs, and pads between the joints, cell membranes, and cartilage. Connective tissue has two components. The chief component is collagen, which is the most common protein in the body, making up one-third of the body's total protein volume. The other component is proteoglycan (PG), which forms the framework for collagenous tissue. PGs are large molecules comprised mainly of glycosaminoglycans (GAGs), which are long chains of modified sugars. Over 30 years of research has gone into understanding how glucosamine acts as a precursor in GAG synthesis.

Performance Parameters—Research has shown that in many cases it has had a positive effect on athletes in both accelerating the healing process and providing relief from joint and cartilage injuries. Scientists have long known that ingesting purified Glucosamine from connective tissue allows the body to bypass the step of converting glucose to Glucosamine. Supplemental Glucosamine clearly aids in the synthesis of connective tissue. All athletes need a supplement that can do this, as the repair and growth of connective tissue is never ending.

Alternative Ingredient—None known. This ingredient because of its solubility in water and taste enhances the color and taste of the drink. It also remains in solution and has no settling properties when chilled or left in solution for long periods of time.

Limitations/Undesirable Characteristics—None known.

Ornithine a-Ketoglutarate (OKG)

Quantity—400 mg

Value/rating—None

Importance—OKG, is the new buzzword being thrown around in American athletic and body building circles. The supplement's popularity is due to the marvelous features it offers the athlete.

Performance Parameters—OKG has been used in Europe for a number of years, as far back as the early 1970s, mainly to treat the victims of burns, trauma, or severe malnutrition and aid post-surgical healing. It seems to be a stimulus for a variety of metabolic functions. It acts as an ammonia scavenger; increases the Glutamine pool in muscle tissue, thereby reducing catabolism; elevates the growth hormone level; increases protein synthesis; increases insulin secretion; and aids L-Glutamine synthesis. Ongoing clinical studies are being conducted on athletes to further understand the potential and effects of the supplement.

Alternative Ingredient—None known. This ingredient because of its solubility in water enhances the color of the drink. It also remains in solution and has no settling properties when chilled or left in solution for long periods of time.

Limitations/Undesirable Characteristics—None known.

Other Ingredients:

Citric Acid

Quantity—(as needed)

Value/rating—None

Importance—A water-soluble natural organic acidulant found widely in citrus plants.

Performance Parameters—Citric Acid is used in this formula to retard the oxidation of fats and oils and to help maintain the clear transparent quality of this drink product. In this formulation it is also used to modify the flavor and to aid in the preservation by lowering the pH level and supporting the orange flavor.

Alternative Ingredient—None. This ingredient is readily soluble in water, which is critical to the clear appearance of the drink. It also remains in solution and will not settle when chilled or left in solution for long periods of time.

Limitations/Undesirable Characteristics—None.

Natural Flavors (orange)

Quantity—(as needed)

Value/rating—None.

Importance—This special combination of natural occurring fruit flavors is formulated, mixed and manufactured specifically for this product.

Performance Parameters—This is one of the most critical components in this product formulation as it functions as a taste-masking ingredient. It also provides a pleasant natural fruity aroma, which corresponds to the designated flavor. In this formulation this ingredient is essential because it solubility which is critical to the color and clear appearance of the drink. It also remains in solution and has no settling properties when chilled or left in solution for long periods of time. Leaves no residue on containers when mixed.

Alternative Ingredient—None. This ingredient is readily soluble in water, which is critical to the clear appearance of the drink. It also remains in solution and will not settle when chilled or left in solution for long periods of time.

Limitations/Undesirable Characteristics—None.

FD&C yellow #6

Quantity—(as required)

Value/rating—None.

Importance—Used as a colorant.

Performance Parameters—Dissolves readily and clearly in water and remains in solution for extended periods of time. Will not settle out of solution when chilled. Leaves no color residue or stain on containers.

Alternative Ingredient—None known. This ingredient because of its solubility in water and coloration delivers the exact color required. It also remains in solution and has no settling properties when chilled or left in solution for long periods of time.

Limitations/Undesirable Characteristics—None know.

Results

Numerous amateur athletes including the inventor have used the inventive formula. The solution is pleasant tasting and consumption following exercise clearly provided hydration and energy and rapid recovery for subsequent exercise. The product was evaluated by two professional athletes training during off-season under the direction of a personal trainer. The athletes reported that consumption of the inventive beverage especially between training episodes allowed them to train significantly longer than other athletic drinks they had been using. Significantly, they reported that loss of weight from excessive exercise was prevented.

The inventive beverage was also used as a training aid during the preseason training of a Midwestern University football team. The trainers were concerned with dehydration and weight loss caused by training. The problem was so great that intravenous administration of fluids was often required. Use of the inventive beverage significantly reduced instances of intravenous fluid administration and significantly prevented weight loss.

The present invention provides a unique formula for replenishing energy stores and for overall recovery following strenuous activity. The combination of straight chain and branched chain (with leucine dominant) amino acids supplement glucose for energy production in muscle tissue during exercise and for tissue repair following exercise. It is believed that the hypotonic combination of salts and glucose provide for rapid and painless absorption of water, salts and carbohydrate. Glucose polymers (maltodextrins) are provided to ensure sustained blood sugar levels. Vitamins ensure maximum metabolic activity for energy production and tissue growth. Vitamins also facilitate metabolism and provide significant antioxidants (both water and lipid soluble). The antioxidant effect is greatly increased Green Tea Extract, Grape Seed Extract and Ferulic Acid. Both Korean and Siberian Ginseng are supplied to enhance energy levels. Ornithine α-ketoglutarate stimulates recovery and tissue building. Glucosamine is provided for the long-term condition of connective tissue and joint lubricity.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

I claim:

1. An aqueous beverage for maximizing endurance and hydration during physical exercise in the form of a substantially optically clear solution wherein a 32 ounce serving comprises:
a carbohydrate source comprising a mixture of glucose polymers, and glucose providing between about 4 and about 12 calories per ounce of said solution;
between about 100 mg and about 300 mg of calcium from a calcium salt;
sodium and potassium from sodium and potassium salts respectively, wherein weight of the sodium is between about ⅔ and about 3 times weight of calcium in the solution;
magnesium and zinc, from magnesium and zinc salts respectively, wherein a weight ratio of calcium to zinc is between about 30:1 and about 10:1, and wherein a weight ratio calcium to magnesium is between about 3:1 to about 1:1;
quantities of vitamin B₁₂ and folate, wherein each quantity is between about 10% and about 300% of recommended daily value;
quantities of vitamins B₁ and B₆, wherein each quantity is between about 50% and about 1,000% of recommended daily value;
quantities of vitamin C and vitamin E, wherein each quantity is between about 50% and 600% of recommended daily value;
a mixture of branched chain and straight chain amino acids, wherein a weight ratio between carbohydrates in the solution and the mixture of amino acids in the solution is between 30:1 and 60:1;
additional antioxidants; and
glucosamine wherein a molar ratio between glucose in the solution and glucosamine in the solution is between about 20:1 and about 120:1.

2. The aqueous beverage for maximizing endurance and hydration during physical exercise according to claim 1, wherein the mixture of branched chain and straight chain amino acids comprises valine, isoleucine and leucine as branched chain amino acids and comprises glutamine and glycine as straight chain amino acids.

3. The aqueous beverage for maximizing endurance and hydration during physical exercise according to claim 2, wherein a molar ratio of straight chain amino acids to branched chain amino acids is about 1.5:1.

4. The aqueous beverage for maximizing endurance and hydration during physical exercise according to claim 2, wherein a molar ratio of glycine to glutamine to valine to isoleucine to leucine is about 2:5:1:1:3.

5. The aqueous beverage for maximizing endurance and hydration during physical exercise according to claim 1, wherein a weight ratio between the carbohydrate source and the mixture of branched chain and straight chain amino acids is between about 30:1 and 60:1.

6. The aqueous beverage for maximizing endurance and hydration during physical exercise according to claim 1, wherein the carbohydrate source further comprises sucrose.

7. The aqueous beverage for maximizing endurance and hydration during physical exercise according to claim 1, further comprising at least 10% of the daily value of chromium.

8. The aqueous beverage for maximizing endurance and hydration during physical exercise according to claim 1, further comprising at least 50 mg per serving of ornithine α-ketoglutarate.

9. The aqueous beverage for maximizing endurance and hydration during physical exercise according to claim 1, wherein the additional antioxidants are selected from the group consisting of green tea leaf extract, grape seed extract, ferulic acid, and mixtures thereof.

10. The aqueous beverage for maximizing endurance and hydration during physical exercise according to claim 1, wherein the carbohydrate source provides about 8 calories per ounce of said solution.

11. The aqueous beverage for maximizing endurance and hydration during physical exercise according to claim 10, wherein the molar ratio between glucose in the solution and glucosamine in the solution is between about 20:1 and 120:1.

12. The aqueous beverage for maximizing endurance and hydration during physical exercise according to claim 1, further comprising a quantity of vitamin A between 10% and 300% of the recommended daily value.

13. The aqueous beverage for maximizing endurance and hydration during physical exercise according to claim 1, wherein about three times the recommended daily value of vitamin C is provided.

14. The aqueous beverage for maximizing endurance and hydration during physical exercise according to claim 1, wherein about twice the recommended daily value of vitamin E is provided.

15. The aqueous beverage for maximizing endurance and hydration during physical exercise according to claim 1, wherein about one and one half times the recommended daily value of vitamin B₁₂ is provided.

16. The aqueous beverage for maximizing endurance and hydration during physical exercise according to claim 1, wherein about all of the recommended daily value of folate is provided.

17. The aqueous beverage for maximizing endurance and hydration during physical exercise according to claim 1, wherein about 200 mg of calcium is provided.

18. The aqueous beverage for maximizing endurance and hydration during physical exercise according to claim 1, wherein about 195 mg of sodium is provided.

19. The aqueous beverage for maximizing endurance and hydration during physical exercise according to claim 18, wherein the weight ratio between sodium and potassium is about 2:3.

20. The aqueous beverage for maximizing endurance and hydration during physical exercise according to claim 1, wherein the weight ratio of calcium to zinc is about 20:1.

21. The aqueous beverage for maximizing endurance and hydration during physical exercise according to claim 1, wherein a weight ratio between calcium to magnesium to zinc to sodium to potassium is about 20:12:1:60:30.

22. An aqueous beverage for maximizing endurance and hydration during physical exercise in the form of a substantially optically clear solution wherein a 32 ounce serving comprises:
a carbohydrate source comprising a mixture of glucose polymers, and glucose providing between about 4 and about 12 calories per ounce of said solution;
between about 100 mg and about 300 mg of calcium from a calcium salt;
sodium and potassium from sodium and potassium salts respectively, wherein weight of the sodium is between about ⅔ and about 3 times weight of calcium in the solution;
magnesium from magnesium salts, wherein a weight ratio of calcium to magnesium is between about 3:1 to about 1:1;
quantities of vitamin C and vitamin E, wherein each quantity is between about 50% and 600% of recommended daily value; and
a mixture of branched chain and straight chain amino acids

23. The aqueous beverage for maximizing endurance and hydration during physical exercise according to claim 22, wherein, weight of the sodium is between about ½ times and about 2 times weight of calcium in the solution.

24. The aqueous beverage for maximizing endurance and hydration during physical exercise according to claim 22, further comprising zinc from zinc salts.

25. The aqueous beverage for maximizing endurance and hydration during physical exercise according to claim 24, wherein a weight ratio of magnesium to zinc is between about 1:1 and about 2:1.

26. The aqueous beverage for maximizing endurance and hydration during physical exercise according to claim 22, further comprising a quantity of vitamin B₁₂.

27. The aqueous beverage for maximizing endurance and hydration during physical exercise according to claim 26, wherein the quantity of vitamin B₁₂ is between about 10% and about 300% of recommended daily value.

28. The aqueous beverage for maximizing endurance and hydration during physical exercise according to claim 22, further comprising a quantity of folate.

29. The aqueous beverage for maximizing endurance and hydration during physical exercise according to claim 28, wherein the quantity of folate is between about 10% and about 300% of recommended daily value.

30. The aqueous beverage for maximizing endurance and hydration during physical exercise according to claim 22 further comprising vitamin B₁.

31. The aqueous beverage for maximizing endurance and hydration during physical exercise according to claim 30, wherein the vitamin B₁ is between about 50% and about 1,000% of recommended daily value.

32. The aqueous beverage for maximizing endurance and hydration during physical exercise according to claim 22 further comprising vitamin B₆.

33. The aqueous beverage for maximizing endurance and hydration during physical exercise according to claim 32 wherein the vitamin B₆ is between about 50% and about 1,000% of recommended daily value.

34. The aqueous beverage for maximizing endurance and hydration during physical exercise according to claim 22, wherein a weight of branched chain amino acids approximately equals a weight of straight amino acids.

35. The aqueous beverage for maximizing endurance and hydration during physical exercise according to claim 22, wherein the straight chain amino acids comprise glycine and glutamine.

36. The aqueous beverage for maximizing endurance and hydration during physical exercise according to claim 22, wherein the branched chain amino acids comprise valine, leucine and isoleucine.

37. The aqueous beverage for maximizing endurance and hydration during physical exercise according to claim 22, further comprising glucosamine.

38. The aqueous beverage for maximizing endurance and hydration during physical exercise according to claim 22, wherein the carbohydrate source further comprises sucrose.

39. The aqueous beverage for maximizing endurance and hydration during physical exercise according to claim 22, further comprising at least 10% of the daily value of chromium.

40. The aqueous beverage for maximizing endurance and hydration during physical exercise according to claim 22, further comprising at least 50 mg per serving of ornithine α-ketoglutarate.

41. The aqueous beverage for maximizing endurance and hydration during physical exercise according to claim 22 further comprising additional antioxidants.

42. The aqueous beverage for maximizing endurance and hydration during physical exercise according to claim 41, wherein the additional antioxidants are selected from the group consisting of green tea leaf extract, grape seed extract, ferulic acid, and mixtures thereof.

43. The aqueous beverage for maximizing endurance and hydration during physical exercise according to claim 22, further comprising a quantity of vitamin A.

44. The aqueous beverage for maximizing endurance and hydration during physical exercise according to claim 43, wherein the quantity of vitamin A is between about 10% and about 300% of recommended daily value.

45. An aqueous beverage for maximizing endurance and hydration during physical exercise in the form of a substantially optically clear solution wherein a 32 ounce serving comprises:
a carbohydrate source comprising a mixture of glucose polymers, glucose and sucrose, wherein about at least half of the carbohydrate source is glucose polymers and a majority of a remaining portion of the carbohydrate source is glucose;
about 200 mg of calcium from a calcium salt;
about 195 mg of sodium and 285 mg of potassium from sodium and potassium salts respectively;
about 120 mg of magnesium and 10 mg of zinc, from magnesium and zinc salts respectively;
vitamin B₁₂ providing about 150% of recommended daily value;
folate providing about 100% of recommended daily value;
vitamin B₁ providing about 666% of recommended daily value;
vitamin B₆ providing about 400% of recommended daily value;
vitamin C providing about 300% of recommended daily value;
vitamin E providing about 200% of recommended daily value;
vitamin A providing about 100% of recommended daily value;
a mixture of branched chain and straight chain amino acids;
additional antioxidants;
botanicals; and
a quantity of glucosamine hydrochloride.

46. The aqueous beverage for maximizing endurance and hydration during physical exercise according to claim 45, wherein the carbohydrate source provides about eight calories per ounce of said solution.

47. The aqueous beverage for maximizing endurance and hydration during physical exercise according to claim 45, wherein the mixture of branched chain and straight chain amino acids comprises about 700 mg of glutamine, about 175 mg of glycine, about 175 mg of valine, about 175 mg of isoleucine and about 350 mg of leucine.

48. The aqueous beverage for maximizing endurance and hydration during physical exercise according to claim 45, wherein the additional antioxidants comprise about 50 mg of green tea leaf extract, about 25 mg of grape seed extract and about 50 mg of ferulic acid.

49. The aqueous beverage for maximizing endurance and hydration during physical exercise according to claim 45, wherein the botanicals comprise about 75 mg of Siberian ginseng and about 150 mg of Koran ginseng.

50. The aqueous beverage for maximizing endurance and hydration during physical exercise according to claim 45 further comprising about 80% of the recommended daily value of chromium.

51. The aqueous beverage for maximizing endurance and hydration during physical exercise according to claim 45, wherein the quantity of glucosamine hydrochloride has a weight of about 300 mg.

52. A method of ensuring rapid recovery following exercise comprising the step of consuming one or more servings of the aqueous beverage according to claim 45.

53. A method of ensuring rapid recovery following exercise comprising the step of consuming one or more servings of the aqueous beverage according to claim 22.

54. A method of ensuring rapid recovery following exercise comprising the step of consuming one or more servings of the aqueous beverage according to claim 1.
